(19) United States
(12) Patent Application Publication
(10) Pub. No.: US 2022/0105275 A1
(43) Pub. Date: Apr. 7, 2022

(54) AUTOMATIC INJECTION SYRINGE

(71) Applicant: Kaiyang JIANG, Nanyang City (CN)

(72) Inventor: Kaiyang JIANG, Nanyang City (CN)

(21) Appl. No.: 17/400,977

(22) Filed: Aug. 12, 2021

(30) Foreign Application Priority Data
Oct. 1, 2020 (CN) 202011093347.7

(51) Int. Cl. A61M 5/315; A61M 5/20

(52) U.S. Cl. CPC A61M 5/31515; A61M 5/31581; A61M 5/20

(57) ABSTRACT

Disclosed is an automatic injection syringe. A telescopic push rod driven by a motor is installed in the syringe, and the medicine injection tube is detachably placed in the syringe; the telescopic push rod comprises a rear rod, a fixed disc, a lead screw assembly, a sliding disc and a front rod, one end of the rear rod is installed on an inner cavity rear end cover of the syringe, the other end of the rear rod is fixedly connected with the fixed disc, the transmission end of the lead screw assembly is installed on the fixed disc, the sliding disc is fixed to nuts of the lead screw assembly, one end of the front rod is fixed to the sliding disc, and the other end of the front rod is inserted into a piston piece at the top of the medicine injection tube in a matched mode.

### PRIORITY

This Application claims priority to pending Chinese application ser. no. 202111093347.7, filed Oct. 1, 2020.

### TECHNICAL FIELD

The present disclosure relates to the technical field of injection syringes, in particular to an automatic injection syringe.

### BACKGROUND

Most of injection syringes used clinically at present are syringe type injection syringes, and liquid medicine in a syringe is injected into a human body by pushing a piston with hands. In the prior art, negative pressure is formed in a syringe body of the syringe by pulling a piston rod with hands, and injection of the injection syringe is completed. A nurse needs to dispense medicine for infusion of a large number of patients every day, and hands are too tired with an original injection syringe; and when treatment injection is carried out in some specialized departments, only one hand can be released since two hands cannot be released, for example, when a doctor in the pain department carries out injection treatment, the doctor can hold a B-ultrasonic device with one hand and the injection syringe with one hand, but an assistant is needed, so that the conventional injection syringe is inconvenient to use with one hand.

### SUMMARY

The present disclosure aims to provide an automatic injection syringe so as to solve the problems existing in the prior art, automatic injection administration can be realized, and the safety injection performance of the injection syringe is improved.

In order to achieve the purpose, the present disclosure provides the following scheme: the automatic injection syringe provided by the present disclosure comprises a syringe, a telescopic push rod and a medicine injection tube, the telescopic push rod driven by a motor is installed in the syringe, and the medicine injection tube is detachably placed in the syringe; the telescopic push rod comprises a rear rod, a fixed disc, a lead screw assembly, a sliding disc and a front rod, one end of the rear rod is installed on an inner cavity rear end cover of the syringe, the other end of the rear rod is fixedly connected with the fixed disc, the transmission end of the lead screw assembly is installed on the fixed disc, the sliding disc is fixed to nuts of the lead screw assembly, one end of the front rod is fixed to the sliding disc, and the other end of the front rod is inserted into a piston piece at the top of the medicine injection tube in a matched mode.

Preferably, the syringe comprises a front cover, a syringe body and a rear cover, and the front cover and the rear cover are detachably connected to the two ends of the syringe body in a screwed mode through threads respectively; and the telescopic push rod and the medicine injection tube are placed in the syringe body.

Preferably, an injection cavity for accommodating the medicine injection tube is formed in the front end of the syringe body, the outer wall of the medicine injection tube and the injection cavity are placed in a clearance fit mode, and the length of the injection cavity is larger than that of the medicine injection tube.

Preferably, two grooves are symmetrically formed in the side wall of the injection cavity, two bulges are symmetrically and integrally arranged on the outer wall of the medicine injection tube, and the two bulges are correspondingly inserted into the two grooves.

Preferably, a circular hole is formed in the center of the front cover of the syringe body, and a syringe needle at the front end of the medicine injection tube corresponds to the circular hole in position.

Preferably, a first motor is arranged in the rear cover, and one end of the rear rod stretches out of the syringe body and then is in transmission connection with the first motor.

Preferably, the lead screw assembly comprises three lead screws, the transmission ends of the three lead screws are uniformly distributed in the circumferential direction and installed on the fixed disc through bearings, three installation holes are uniformly formed in the sliding disc in the circumferential direction, the bottom ends of the three lead screws penetrate through the corresponding installation holes respectively, and the nuts of the lead screws are fixed to the installation holes in an interference fit mode.

Preferably, an injection button and a return button are arranged on the outer wall of the syringe, and are both electrically connected with a motor for driving the three lead screws to move; the injection button is used for controlling feeding of the lead screws and driving the front rod to move to push out liquid medicine in the medicine injection tube; and the return button is used for controlling returning of the lead screws and driving the front rod to return so as to drive the medicine injection tube provided with the syringe needle to return.

Preferably, limiting buckles are installed at the bottom ends of the three lead screws respectively.

Preferably, the piston piece at the top of the medicine injection tube is a rubber plug, an inserting hole is formed in the top of the rubber plug, and the tail end of the front rod is inserted into the inserting hole; and on the premise of ensuring the sealing of the rubber plug, the front rod drives the rubber plug to slide up and down relative to the inner wall of the medicine injection tube.

Compared with the prior art, the present disclosure has the following beneficial technical effects:

The automatic injection syringe in the present disclosure is reasonable in structural design, convenient to install and use, capable of achieving automatic injection action of the injection syringe, more stable in action and high in safety coefficient. Injection operation of different patients can be achieved by opening the front cover to replace the medicine injection tube, the risk of cross medicine infection does not exist, and the working efficiency of medical staff is improved.

### BRIEF DESCRIPTION OF THE DRAWINGS

To more clearly illustrate the embodiment of the present disclosure or the technical scheme in the prior art, the following briefly introduces the attached figures to be used in the embodiment. Apparently, the attached figures in the following description show merely some embodiments of the present disclosure, and those skilled in the art may still derive other drawings from these attached figures without creative efforts.

FIG. 1 is an integral structural schematic diagram (a syringe needle being in a return state) of an automatic injection syringe; and

FIG. 2 is a schematic diagram of an injection state of the automatic injection syringe.

Reference signs: 1, back cover; 2, fixed disc; 3, sliding disc; 4, lead screw; 5, limiting buckle; 6, syringe body; 7, medicine injection tube; 8, syringe needle; 9, injection cavity; 10, front cover; 11, rubber plug; 12, front rod; 13, injection button; 14, return button; 15, rear rod; and 16, first motor.

### DETAILED DESCRIPTION

The following clearly and completely describes the technical scheme in the embodiments of the present disclosure with reference to the attached figures in the embodiments of the present disclosure. Apparently, the described embodiments are merely a part rather than all of the embodiments of the present disclosure. All other embodiments obtained by those skilled in the art based on the embodiments of the present disclosure without creative efforts shall fall within the protection scope of the present disclosure.

The present disclosure aims to provide an automatic injection syringe so as to solve the problems existing in the prior art, automatic injection administration can be realized, and the safety injection performance of the injection syringe is improved.

To make the foregoing objective, features and advantages of the present disclosure clearer and more comprehensible, the present disclosure is further described in detail below with reference to the attached figures and specific embodiments.

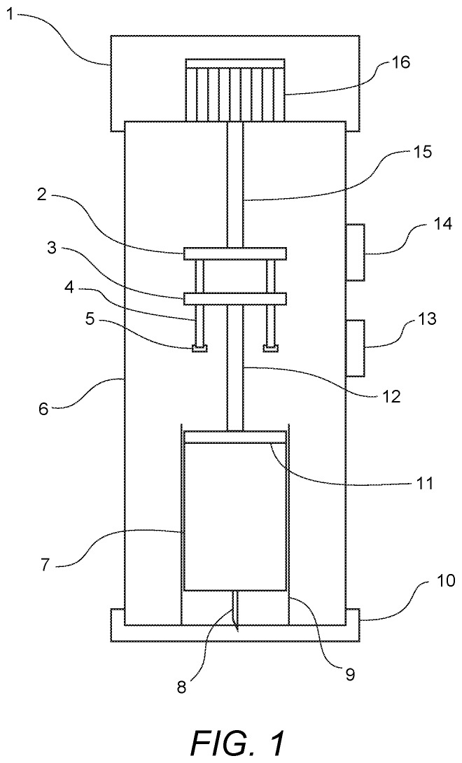

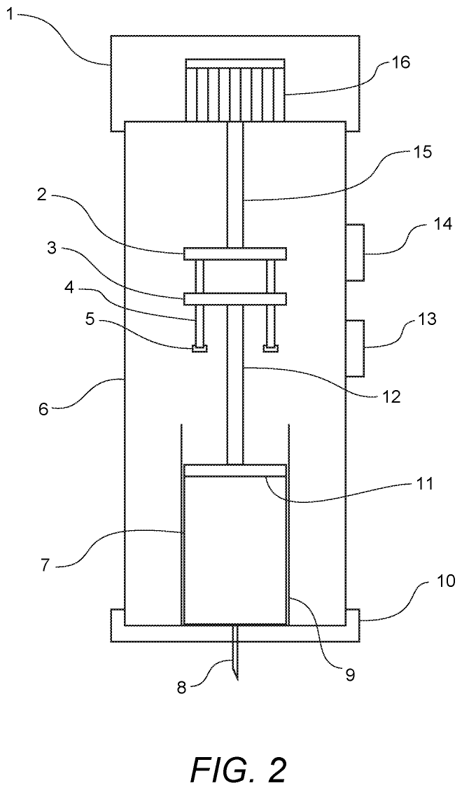

As shown in FIG. 1 and FIG. 2, the embodiment provides the automatic injection syringe comprising a syringe, a telescopic push rod and a medicine injection tube 7, the telescopic push rod driven by a motor is installed in the syringe, and the medicine injection tube 7 is detachably placed in the syringe; the telescopic push rod comprises a rear rod 15, a fixed disc 2, a lead screw assembly, a sliding disc 3 and a front rod 12, one end of the rear rod 15 is installed on an inner cavity rear end cover of the syringe, the other end of the rear rod is fixedly connected with the fixed disc 2, the transmission end of the lead screw assembly is installed on the fixed disc 2, the sliding disc 3 is fixed to nuts of the lead screw assembly, one end of the front rod 12 is fixed to the sliding disc 3, and the other end of the front rod is inserted into a piston piece at the top of the medicine injection tube 7 in a matched mode.

In the using process, the medicine injection tube filled with liquid medicine is placed in the syringe, the tail end of the front rod 12 is inserted into an opening in the top of the medicine injection tube 7, the sliding disc 3 is driven to move downwards through movement of the lead screw assembly, and then the front rod 12 pushes the piston piece to move downwards relative to the medicine injection tube 7 for injection administration.

Specifically, the syringe in the embodiment comprises a front cover 10, a syringe body 6 and a rear cover 1, and the front cover 10 and the rear cover 1 are detachably connected to the two ends of the syringe body 6 in a screwed mode through threads respectively; and the telescopic push rod and the medicine injection tube 7 are placed in the syringe body 6. An injection cavity 9 for accommodating the medicine injection tube 7 is formed in the front end of the syringe body 6, the outer wall of the medicine injection tube 7 and the injection cavity 9 are placed in a clearance fit mode, and the length of the injection cavity 9 is larger than that of the medicine injection tube 7. Firstly, the front cover 10 is unscrewed, then the medicine injection tube 7 filled with liquid medicine is installed in the injection cavity 9, at the moment, the tail end of the front rod 12 is inserted into an inserting hole of the piston piece at the top of the medicine injection tube 7, the front cover 10 can be screwed after the medicine injection tube 7 is installed, and a circular hole in the front cover 10 corresponds to a syringe needle 8.

In order to ensure that the medicine injection tube 7 can be accurately installed in place, two grooves are symmetrically formed in the side wall of the injection cavity 9 in the embodiment, two bulges are symmetrically and integrally arranged on the outer wall of the medicine injection tube 7, and the two bulges are correspondingly inserted into the two grooves. The grooves and the bulges are arranged, so that the medicine injection tube 7 can be conveniently installed and positioned in the injection cavity 9, so that the front rod 12 can be more accurately inserted into the inserting hole.

In order to achieve telescoping of the telescopic push rod, in the embodiment, the lead screw assembly comprises three lead screws 4, the transmission ends of the three lead screws 4 are uniformly distributed in the circumferential direction and installed on the fixed disc 2 through bearings, three installation holes are uniformly formed in the sliding disc 3 in the circumferential direction, the bottom ends of the three lead screws 4 penetrate through the corresponding installation holes respectively, and the nuts of the lead screws 4 are fixed to the installation holes in an interference fit mode. An injection button 13 and a return button 14 are arranged on the outer wall of the syringe, and are both electrically connected with a motor for driving the three lead screws to move; the injection button 13 is used for controlling feeding of the lead screws 4 and driving the front rod 12 to move to push out liquid medicine in the medicine injection tube 7; and the return button 14 is used for controlling returning of the lead screws 4 and driving the front rod 12 to return so as to drive the medicine injection tube 7 provided with the syringe needle 8 to return.

The working process of the automatic injection syringe is as follows: after the medicine injection tube 7 is installed and the front cover 10 is screwed, the injection button 13 is pressed down, the motor drives the lead screws 4 to rotate, the nuts on the lead screws 4 drive the sliding disc 3 to feed, the sliding disc 3 moves forwards relative to the lead screws 4, and at the moment, the front rod 12 feeds forwards and drives the medicine injection tube 7 to move forwards while pushing the piston piece to move; when the front end of the medicine injection tube 7 abuts against the front cover 10, the syringe needle 8 stretches out of the front cover 10, at the moment, the medicine injection tube 7 does not move any more, the front rod 12 continues to push the piston piece forwards, and therefore injection of liquid medicine is achieved.

After injection is completed, the return button 14 is pressed down, the motor drives the lead screws 4 to rotate, the nuts on the lead screws 4 drive the sliding disc 3 to return, the sliding disc 3 moves backwards relative to the lead screws 4, at the moment, the front rod 12 returns backwards and drives the piston piece on the medicine injection tube 7 to return together while returning, and therefore the medicine injection tube 7 is driven to move backwards relative to the injection cavity 9, and the syringe needle 8 is pulled out.

Besides, a first motor 16 is arranged in the rear cover 1, one end of the rear rod 15 stretches out of the syringe body 6 and then is in transmission connection with the first motor 16, the first motor 16 can drive the rear rod 15 to rotate, the rear rod 15 can drive the fixed disc 2, the sliding disc 3 and the front rod 12 to rotate while rotating, and therefore the front rod 12 can be better matched with the piston piece and screwed down.

In order to prevent the sliding disc 3 from being separated from the lead screws 4 in the feeding process, limiting buckles 5 are installed at the bottom ends of the three lead screws respectively.

In the embodiment, the piston piece at the top of the medicine injection tube can be a rubber plug 11 or a sealing push plate, and on the premise that the rubber plug 11 or the sealing push plate and the inner wall of the medicine injection tube 7 are sealed to prevent liquid medicine from overflowing, when the piston piece selects the rubber plug 11, the inner diameter of the inserting hole in the rubber plug 11 is slightly smaller than the outer diameter of the front rod 12; in this way, the front rod 12 can be tightly inserted into the rubber plug 11 while pushing the piston piece to be fed, and therefore it is guaranteed that the piston piece can return along with the front rod 12 in the returning process; and meanwhile, a limiting clamp is also arranged at the top end of the medicine injection tube 7 to prevent the piston piece from separating from a tube body. When the piston piece selects the sealing push plate, a connecting hole with internal threads is formed in the top of the push plate, the rear rod 15 rotates to drive external threads of the front rod 12 to be connected with internal threads of the push plate in the matched mode, feeding pushing can be achieved, and it can be guaranteed that the push plate returns along with the front rod 12.

In the embodiment, a storage battery is arranged in the automatic injection syringe, the storage battery is used for supplying power to the motor and a driving part in the injection syringe under the condition that the electricity of the storage battery is sufficient, and when the electricity of the storage battery is low, the storage battery can be charged through a wiring socket, so that subsequent injection operation is facilitated.

For those skilled in the art, obviously the present disclosure is not limited to the details of the exemplary embodiment, and the present disclosure can be achieved in other specific forms without departing from the spirit or essential characteristics of the present disclosure. Therefore, for every point, the embodiments should be regarded as exemplary embodiments and are unrestrictive, the scope of the present disclosure is restricted by the claims appended hereto, therefore, all changes, including the meanings and scopes of equivalent elements, of the claims are aimed to be included in the present disclosure, and any mark of attached figures in the claims should not be regarded as limitation to the involved claims.

Several examples are used for illustration of the principles and implementation methods of the present disclosure. The description of the embodiments is used to help illustrate the method and core principles of the present disclosure; and in addition, those skilled in the art can make various modifications in terms of specific embodiments and scope of application in accordance with the thoughts of the present disclosure. In conclusion, the content of this specification shall not be construed as a limitation to the present disclosure.

What is claimed is:

1. An automatic injection syringe, comprising:
a syringe, a telescopic push rod and a medicine injection tube, wherein the telescopic push rod driven by a motor is installed in the syringe, and the medicine injection tube is detachably placed in the syringe;
the telescopic push rod comprises a rear rod, a fixed disc, a lead screw assembly, a sliding disc and a front rod;
wherein one end of the rear rod is installed on an inner cavity rear end cover of the syringe, the other end of the rear rod is fixedly connected with the fixed disc, the transmission end of the lead screw assembly is installed on the fixed disc, the sliding disc is fixed to nuts of the lead screw assembly, one end of the front rod is fixed to the sliding disc, and the other end of the front rod is inserted into a piston piece at the top of the medicine injection tube in a matched mode.

2. The automatic injection syringe according to claim 1, wherein the syringe comprises a front cover, a syringe body and a rear cover, and the front cover and the rear cover are detachably connected to the two ends of the syringe body in a screwed mode through threads respectively; and the telescopic push rod and the medicine injection tube are placed in the syringe body.

3. The automatic injection syringe according to claim 2, wherein an injection cavity for accommodating the medicine injection tube is formed in the front end of the syringe body, the outer wall of the medicine injection tube and the injection cavity are placed in a clearance fit mode, and the length of the injection cavity is larger than that of the medicine injection tube.

4. The automatic injection syringe according to claim 3, wherein first and second grooves are symmetrically formed in the side wall of the injection cavity, two bulges are symmetrically and integrally arranged on the outer wall of the medicine injection tube, and the two bulges are correspondingly inserted into the first and second grooves.

5. The automatic injection syringe according to claim 2, wherein a circular hole is formed in the center of the front cover of the syringe body, and a syringe needle at the front end of the medicine injection tube corresponds to the circular hole in position.

6. The automatic injection syringe according to claim 2, wherein a first motor is arranged in the rear cover, and one end of the rear rod stretches out of the syringe body, and is then in transmission connection with the first motor.

7. The automatic injection syringe according to claim 1, wherein the lead screw assembly comprises three lead screws, the transmission ends of the three lead screws are uniformly distributed in the circumferential direction and installed on the fixed disc through bearings, three installation holes are uniformly formed in the sliding disc in the circumferential direction, the bottom ends of the three lead screws penetrate through the corresponding installation holes respectively, and the nuts of the lead screws are fixed to the installation holes in an interference fit mode.

8. The automatic injection syringe according to claim 7, wherein an injection button and a return button are arranged on the outer wall of the syringe, and are both electrically connected with a motor for driving the three lead screws to move; the injection button is used for controlling feeding of the lead screws and driving the front rod to move to push out liquid medicine in the medicine injection tube; and the return button is used for controlling returning of the lead screws and driving the front rod to return so as to drive the medicine injection tube provided with the syringe needle to return.

9. The automatic injection syringe according to claim 7, wherein limiting buckles are installed at the bottom ends of the three lead screws respectively.

10. The automatic injection syringe according to claim 1, wherein the piston piece at the top of the medicine injection tube is a rubber plug, an inserting hole is formed in the top of the rubber plug, and the tail end of the front rod is inserted into the inserting hole; and on the premise of ensuring the sealing of the rubber plug, the front rod drives the rubber plug to slide up and down relative to the inner wall of the medicine injection tube.
